(19) United States
(12) Patent Application Publication
(10) Pub. No.: US 2007/0270835 A1
(43) Pub. Date: Nov. 22, 2007

(54) Bone attachment devices with a threaded interconnection including a solid lubricious material

(75) Inventor: Paul J. Wisnewski, Maple Grove, MN (US)

Correspondence Address: KRIEG DEVAULT LLP, ONE INDIANA SQUARE, SUITE 2800, INDIANAPOLIS, IN, 46204-2709, US

(73) Assignee: SDGI Holdings, Inc.

(21) Appl. No.: 11/429,037

(22) Filed: May 5, 2006

(51) Int. Cl. A61F 2/30

(52) U.S. Cl. 606/61

(57) ABSTRACT

One embodiment of the present application includes an apparatus comprising a bone attachment device for a spinal fixation system or other implant arrangement. The device has a socket with threading, with the socket defining a channel to accept a system component to be secured therein by engaging the threading with a threaded fastener. A solid lubricious material is applied between the threading and the threaded fastener to facilitate easier installation, increased locking forces, and decreased distortion of system components. The lubricious material may be affixed to one or more the threaded components to be separately carried therewith prior to interconnection. Other implant apparatus, systems, processes, and techniques are also disclosed.

### BACKGROUND

The present application relates to a prosthetic device and a manner of using and making the same, and more specifically, but not exclusively, concerns increasing the locking strength of the threaded interconnection of a biomechanical construct for the spine.

The use of prosthetic implant devices to address orthopedic injuries has become commonplace. In this arena, it is often desired to decrease the invasiveness of the procedures, improve implant integrity, and provide more positive patient outcomes. Some implant devices utilize threaded interconnections of components to provide a stable construct. However, there is still some room for further improvement of the mechanical integrity of such devices, resulting in a need for continued contributions in this technical area.

### SUMMARY

One embodiment of the present application is a unique implantable construct. Other embodiments include unique methods, systems, devices, instrumentation, and apparatus involving an orthopedic prosthesis.

A further embodiment of the present application includes a multiaxial bone screw device having a threaded portion which is designed to engage a bone or bony structure. This multiaxial screw further has a head with a socket designed for receiving an orthopedic rod. In one form, the rod may be designed to extend through the socket region to allow for interconnection with one or more other components. The socket of the head defines threading structured for engagement by a threaded fastener. When the rod is received in the socket, the threaded fastener fixes the rod to the head, with the rod being positioned between the fastener and the socket. In order to reduce friction between the threading and the fastener and to facilitate easier application, a solid lubricious coating is utilized. In some forms, the coating may be affixed to the threading of the head, the threaded fastener, or both. In still further forms, the lubricious solid material can be applied between the head and fastener threading as a separate component.

Another embodiment of the present application includes: making a multiaxial bone screw including a longitudinal stem with a threaded portion structured to anchor to bone and a head attached to the stem, the head including a threaded socket; making a threaded fastener structured to engage the threaded socket to provide an implant construct; and at least partially coating one or more of the threaded socket and the threaded fastener with a solid lubricious material.

Still another embodiment includes engaging a bone attachment device to a bone at a desired skeletal location. In one particular form, the bone belongs to the spine. This embodiment also includes inserting a rod into a channel area of a saddle member connected to the bone attachment device. The channel member includes a pair of upright sections having threading. A threaded fastener engages the threading to secure the rod to the saddle member. The threaded fastener includes a coating of solid lubricious material affixed thereto. Alternatively or additionally, a coating of solid lubricious material is affixed to the threading of the saddle member.

Yet a further embodiment of the present application includes a bone attachment device that may be, but is not limited to, a bone screw or a hook. The device includes a saddle member having a pair of upright members forming a channel through which a rod may extend. The upright members forming the channel define threading. A threaded fastener is provided to engage the threading to fix the rod received in the channel to the bone attachment device. In a further embodiment, a solid lubricious coating is affixed to the threaded fastener and/or the threading.

One object of the present application is to provide a unique prosthesis.

Alternatively or additionally, another object of the present application is to provide a unique orthopedic prosthetic method, system, device, instrument, kit and/or apparatus.

Further embodiments, forms, features, aspects, benefits, objects, and advantages of the present application shall become apparent from the detailed description and figures provided herewith.

### BRIEF DESCRIPTION OF THE DRAWING

FIG. 1 is a posterior view of a spinal fixation device relative to the spinal column of a patient.

FIG. 2 is a partial sectional, exploded assembly view of a multiaxial bone screw device included in the system of FIG. 1. The device includes a fastener for which coating layer thickness has been exaggerated to enhance clarity.

FIG. 3 is a partial sectional, view of the multiaxial bone screw device of FIG. 2 assembled together.

FIG. 4 is a top view of a threaded fastener for the multiaxial bone screw device of FIGS. 2 and 3.

FIG. 5 is a perspective view of another type of bone attachment device.

FIG. 6 is a top view of a bone attachment device and rod prior to assembly with a threaded fastener.

FIG. 7 is a top view of the bone attachment device and rod of FIG. 6 after assembly with a threaded fastener.

### DETAILED DESCRIPTION OF REPRESENTATIVE EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

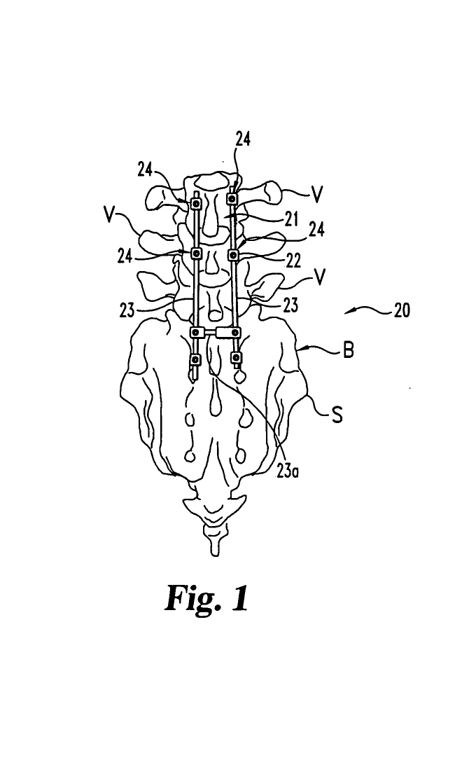

FIG. 1 illustrates a posterior spinal fixation system 20 of one embodiment of the application located at a desired skeletal location of a patient. More specifically, as depicted in FIG. 1, system 20 is affixed to bones B of the spinal column 21 from a posterior approach. Bones B include the sacrum S and several vertebrae V. System 20 generally includes several bone attachment devices 22 and rods 23 structured to selectively interconnect with bone attachment devices 22. While shown with an approximately circular cross section, rod 23 may be differently shaped in alternative embodiments. Rod 23 may be solid or hollow along some or all of its length and/or may be of homogenous or heterogeneous composition. In system 20, bone attachment devices 22 are affixed to various locations of the spinal column 21 and interconnected with rods 23 that are, in turn, interconnected by a lateral bridge member 23a to provide a stable construct for treating spinal disorders. Posterior fixation system 20 may be used for, but is not limited to, treatment of degenerative spondylolisthesis, fracture, dislocation, scoliosis, kyphosis, spinal tumor, and/or a failed previous fusion.

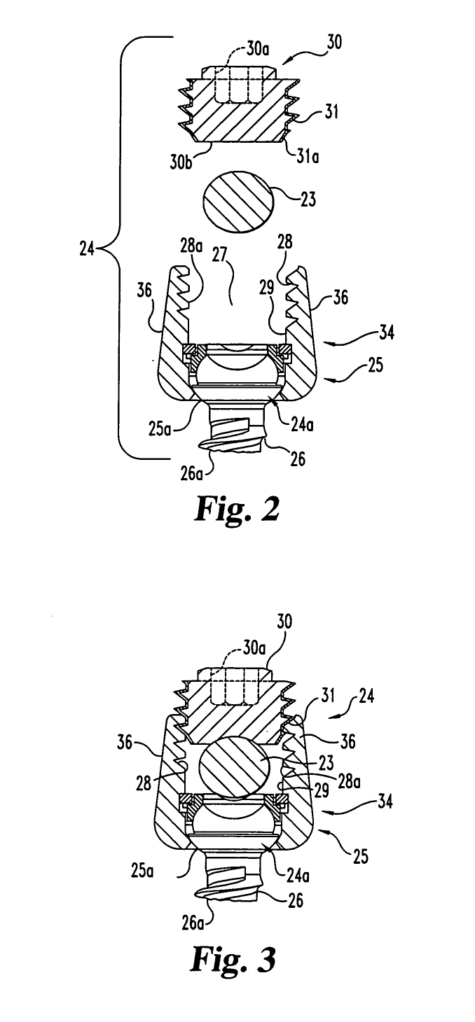

One type of bone attachment device 22 included in system 20 is a multiaxial bone screw assembly 24. FIGS. 2 and 3 provide exploded assembly and assembled sectional views, respectively, of multiaxial bone screw assembly 24 in greater detail; where like reference numerals refer to like features previously described. In one form, multiaxial bone screw assembly 24 comprises medical grade stainless steel but other embodiments may comprise, but are not limited to, titanium, a titanium alloy or other metallic alloy, and/or a nonmetallic composition. Multiaxial bone screw assembly 24 includes a longitudinal bone screw 24a with a head 25. Head 25 is alternatively designated saddle member 34. Multiaxial bone screw 24a includes a longitudinal threaded stem 26. A helical threaded portion 26a of stem 26 is only partially illustrated in FIGS. 2 and 3, it being understood that helical threading continues along a desired length of screw 24a in a standard manner (not shown). Stem 26 is structured to threadingly engage a passageway prepared through one or more bones or bone fragments in a standard manner. Stem 26 and head 25 are engaged together with a ball-and-joint or “swivel” type of coupling 25a that permits relative movement between stem 26 and head 25 to adjustably position screw 24a relative to head 25 and rod 23 before rigidly fixing them together.

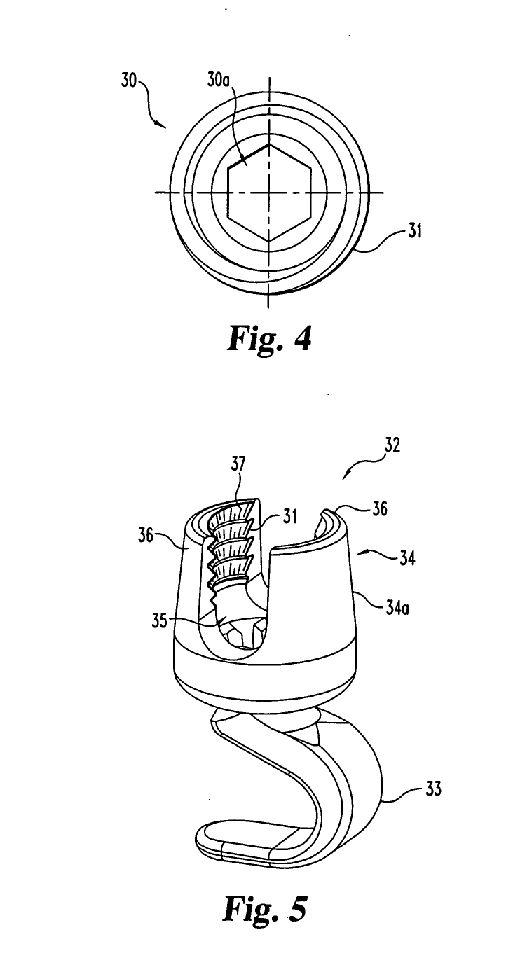

Head 25 includes a socket 27 defined by opposing upright portions 36. Socket 27 includes socket threading 28 and defines a channel 29 therethrough. Socket 27, and more particularly channel 29, is designed to receive one of rods 23. In various embodiments of this application, socket 27 and rod 23 may differ in size in relation to one another and/or other components of system 20. Assembly 24 further includes threaded fastener 30. Threaded fastener 30 is designed to engage socket threading 28 by rotation into socket 27. As depicted, fastener 30 is in the form of a set screw that includes a cavity 30a for tool engagement. In the depicted embodiment, cavity 30a is of a hex or allen wrench shape. In alternative embodiments, cavity 30a may be differently shaped for engagement by an appropriate assembly tool or may be absent. Indeed, in one alternative, fastener 30 includes a frangible, break-away portion which is proximal relative to the fastener threading. For this alternative fastener 30 is threaded into socket 27 until a threshold torque level is reached, at which point the proximal break-away portion fractures, separating from the fastener at a point above its engagement in socket 27. Referring also to FIG. 4, a top view of fastener 30 is provided that further illustrates cavity 30a; where like reference numerals refer to like features previously described. In FIG. 4, fastener 30 is shown without the other components of assembly 24 and system 20 to preserve clarity.

Opposite cavity 30a, fastener 30 includes bearing end portion 30b that is structured to contact rod 23 when assembled together as best shown in FIG. 3. Multiaxial bone screw 24a and head 25 are coupled together in a standard manner to permit movement relative to one another with multiple rotational degrees of freedom before assembly with rod 23 and fastener 30. Rod 23, head 25, and end portion 30b become fixed together to provide a rigid construct when fastener 30 is threaded into socket 27 and sufficiently tightened therein. In turn, as rod 23 bears against coupling 25a and stem 26 with the tightening of fastener 30, the position of stem 26 relative to head 25 becomes fixed and rigid. Such aspects are more fully described in commonly owned U.S. Pat. No. 6,485,491 to Farris et al., which is hereby incorporated by reference. In one embodiment described in this reference, multiaxial bone screw member 24a includes an expansion member which expands to lock the position of multiaxial bone screw 24a and head 25 together.

As perhaps best shown in the sectional view of FIG. 2, threaded fastener 30 further includes a coating 31 in contact with at least a portion of its threaded surface 31a. The thickness of coating 31 is exaggerated in FIG. 2 to enhance clarity. Coating 31 is comprised of a solid lubricious material that increases the lubricity of the mating surfaces of threaded fastener 30 and socket threading 28 to facilitate an easier and more secure assembly. More specifically, it has been surprisingly discovered that the increased lubricity of coating 31 relative to the surface it coats and/or engages, provides a greater locking force between threaded fastener 30 with socket threading 28 than would occur in the absence of coating 31 under the same applied torque.

The solid lubricious material of coating 31 has a coefficient of friction less than that of the material defining surface 31a of fastener 30 and/or surface 28a of head 25 that defines socket threading 28. Coating 31 is also in a form which will not be substantially expelled from the contact area of threaded fastener 30 and socket threading 28 when engaged to one another. In preferred embodiments, coating 31 may include, but is not limited to, a metallic material, a polymeric material, or a ceramic material. In one more preferred embodiment utilizing a metallic coating material, coating 31 is chrome. In another more preferred embodiment utilizing a ceramic coating material, coating 31 is a diamond-like carbon-based coating. In still another more preferred embodiment, coating 31 is a polymeric material that includes at least one of polytetraflouroethylene (PTFE) or parylene. In an even more preferred embodiment, coating 31 is parylene. Parylene is desirable in at least some applications because of its biocompatibility and cost effectiveness. Additionally, application of parylene as coating 31 to threaded fastener 30 can be desirable from a manufacturing perspective because it may be applied at room temperature by a gas deposition process which facilitates coating thickness control. Nonetheless, in other embodiments a different coating composition and/or manufacturing technique can be utilized. Further, a lubricious material of any of these types and/or another type can be applied to threading 28 of socket 27 prior to engagement with fastener 30 as an alternative to coating 31 of fastener 30 or in addition to the application of coating 31 on fastener 30.

Another type of bone attachment device 22 is more specifically illustrated in FIG. 5 as bone anchor 32; where like reference numerals refer to like features previously. Bone anchor 32 can be comprised of the same materials as one or more components of assembly 24 or may differ as would occur to those skilled in the art. Bone anchor 32 includes a bone engagement hook 33 of a standard type and a saddle member 34. Saddle member 34 includes a head 34a with upright portions 36 defining a channel 35 therethrough. Upright portions 36 each define threading 37. Channel 35 is shaped and sized for acceptance of additional spinal fixation device components, such as rod 23 and fastener 30. In one arrangement, rod 23 is secured in channel 35 by threaded interconnection of fastener 30 with threading 37 of head 34a. In the depicted embodiment, threading 37 is at least partially covered by coating 31 of the type previously described in connection with assembly 24. It should be appreciated that in various embodiments, coating 31 or another lubricant may be present on both threading 37 and fastener 30 or just one or the other.

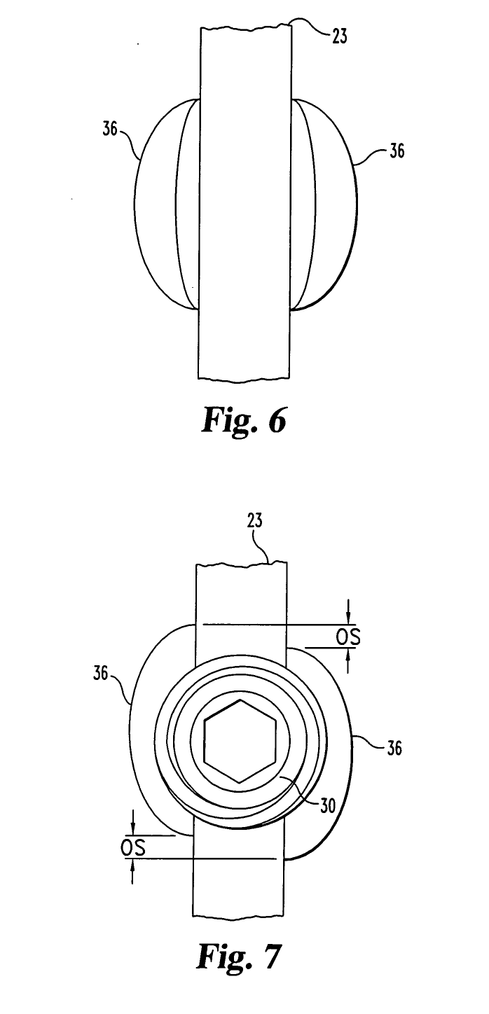

FIGS. 6 and 7 illustrate different stages on construct assembly without a solid lubricious material (such as that provided by coating 31); where like reference numerals refer to like features previously described. FIG. 6 is a top view of rod 23 situated between upright portions 36. The interior of each upright portion 36 includes threading of the type described in connection with FIG. 2, 3, or 5 that is structured for engagement by a threaded fastener (such as fastener 30); however, the threaded fastener is not present in the FIG. 6 view.

FIG. 7 is a top view of rod 23 situated between upright portions 36, as depicted in FIG. 6, but after the threaded fastener without a lubricious coating 31 has been assembled therewith. In the absence of such materials, frictional engagement of upright portions 36 with the fastener can cause the upright portions 36 to distort as the fastener is tightened. This distortion can deform upright portion(s) 36, resulting in an asymmetry such as that corresponding offset OS shown in FIG. 7. In some applications, it is desirable to lessen this distortion or provide greater locking force than would otherwise be provided by components with undesirably high friction coefficients. In such applications, it has been surprisingly discovered that a solid lubricious coating as previously described, can satisfy such desires, as appropriate.

In one experimental example, testing was conducted by Medtronic Sofamor Danek with parylene-coated threaded fasteners of the type shown in FIGS. 2 and 3. This testing showed a significant increase in slip load of the parylene-coated threaded fasteners. In one trial, the fastener had an 8 millimeter (mm) diameter and was evenly coated with approximately 0.0005 inch of parylene. This parylene coating had a coefficient of friction of about 0.3. The testing measured the force required to cause motion of the threaded fastener inside the connector. An uncoated threaded fastener had a slip load of 340 N (SD 79N) while the parylene coated threaded fastener had a slip load of 406 N (SD 22N). These results indicate a greater likelihood of higher and more consistent locking loads under actual operating conditions.

While the invention has been illustrated and described in detail in the drawings and foregoing description, the same is to be considered illustrative and not restrictive in character, it being understood that only selected embodiments have been shown and described and that all changes, equivalents, and modifications that come within the scope of the inventions described herein or defined by the following claims are desired to be protected. Any experiments, experimental examples, or experimental results provided herein are intended to be illustrative of the present invention and should not be construed to limit or restrict the invention scope. Further, any theory, mechanism of operation, proof, or finding stated herein is meant to further enhance understanding of the present invention and is not intended to limit the present invention in any way to such theory, mechanism of operation, proof, or finding. In reading the claims, words such as “a”, “an”, “at least on”, and “at least a portion” are not intended to limit the claims to only one item unless specifically stated to the contrary. Further, when the language “at least a portion” and/or “a portion” is used, the claims may include a portion and/or the entire item unless specifically stated to the contrary.

What is claimed is:

1. An apparatus, comprising:
a multiaxial bone screw device including a longitudinal threaded stem structured to engage bone and a head, the head defining a socket with threading;
a rod structured to extend through the socket;
a threaded fastener structured to engage the threading of the socket to fix the rod in the socket between the fastener and the stem; and
wherein a coating is affixed to one or more of the threading and the threaded fastener to be carried therewith when spaced apart from one another, the coating including a solid lubricious material to reduce friction between the threaded fastener and the threading of the socket when the threaded fastener engages the threading of the socket to secure the rod therein.

2. The apparatus in claim 1, wherein the coating has a coefficient of friction less than one or more of a material of the threading and a material of the threaded fastener when said coating is absent from said material of said threading and said material of said threaded fastener.

3. The apparatus in claim 2 wherein the solid lubricious material consists of at least one of a metallic material, a polymeric material, and an amorphous material.

4. The apparatus in claim 3 wherein the metallic material comprises chrome plating.

5. The apparatus in claim 3 wherein the polymeric material includes one or more of poly-tetraflouroethylene and parylene.

6. The apparatus in claim 3 wherein the amorphous material comprises diamond-like carbon.

7. A method, comprising:
making a multiaxial bone screw including a longitudinal stem with a threaded portion structured to anchor to bone and a head attached to the stem, the head including a threaded socket;
making a threaded fastener structured to engage the threaded socket to provide an implant construct; and
at least partially coating one or more of the threaded socket and the threaded fastener with a solid lubricious material.

8. The method of claim 7 wherein the coating includes performing a gas deposition process.

9. The method of claim 8 wherein the solid lubricious material includes at least one of poly-tetraflouroethylene and parylene.

10. The method of claim 7, which includes providing a rod structured to be received in the socket and fixed between the fastener and the socket when the fastener is threaded in the socket;

11. A method, comprising:
engaging a bone attachment device to a bone at a desired skeletal location, the device including a saddle member having a pair of upright portions defining a channel with threading;
placing a rod to extend through the channel;
providing a threaded fastener, at least one of the threading and the threaded fastener being coated with a solid lubricious material carried therewith when spaced apart from one another; and
securing the rod in the channel by engaging the threading of the saddle member with the threaded fastener.

12. The method of claim 11 wherein the bone attachment device includes a longitudinal threaded stem structured to engage the bone, the threaded stem being positioned opposite the saddle member.

13. The method of claim 11 wherein the bone attachment device includes a hook to contact the bone, the hook being positioned opposite the saddle member.

14. The method of claim 11 wherein the solid lubricious material consists of at least one of a metallic material, polymeric material, and an amorphous material.

15. The method of claim 14 wherein the polymeric material includes one or more of poly-tetraflouroethylene and parylene.

16. An apparatus comprising:
a bone attachment device including a saddle member, the saddle member having a pair of upright portions defining a channel with threading;
a rod structured to extend through the channel;
a threaded fastener structured to engage the threading of the channel to secure the rod in the channel between the fastener and the bone attachment device; and
wherein a solid lubricious material at least partially covers one or more of the threading and the threaded fastener to reduce friction between the threading of the saddle member and the threaded fastener when the threaded fastener engages the threading, the coating being affixed to the one or more of the threading and the threaded fastener to be carried therewith.

17. The apparatus of claim 16 wherein the bone attachment device includes a longitudinal threaded stem opposite the saddle member.

18. The apparatus of claim 16 wherein the bone attachment device includes a hook opposite the saddle member.

19. The apparatus of claim 16 wherein the solid lubricious material comprises a metallic coating.

20. The apparatus of claim 19 wherein the metallic coating comprises chrome plating.

21. The apparatus of claim 16 wherein the solid lubricious material comprises polymeric plating.

22. The apparatus of claim 21 wherein the polymeric coating comprises poly-tetraflouroethylene.

23. The apparatus of claim 21 wherein the polymeric coating comprises parylene.

24. The apparatus of claim 16 wherein the solid lubricious material comprises diamond-like carbon.
